(19) United States
(12) Patent Application Publication
(10) Pub. No.: US 2026/0335225 A1
(43) Pub. Date: Oct. 29, 2026

(54) OPHTHALMIC SUSPENSION COMPOSITION OF BRINZOLAMIDE AND LATANOPROST

(71) Applicant: SENTISS PHARMA PRIVATE LIMITED, New Delhi (IN)

(72) Inventors: Lalit Kumar PATEL, New Delhi (IN); Manoj NAHAR, New Delhi (IN); Ajay R. MHASKE, New Delhi (IN); Sukhjeet SINGH, New Delhi (IN); Deepak BAHRI, New Delhi (IN)

(21) Appl. No.: 19/486,076

(22) PCT Filed: May 22, 2024
(86) PCT No.: PCT/IN2024/050597

§ 371 Date: Nov. 20, 2025

(30) Foreign Application Priority Data
May 22, 2023 (IN) 202311035553

(51) Int. Cl. A61K 9/00; A61K 31/542; A61K 31/5575; A61K 47/26; A61K 47/40

(52) U.S. Cl. CPC A61K 9/0048; A61K 31/542; A61K 31/5575; A61K 47/26; A61K 47/40

(57) ABSTRACT

The present invention provides an ophthalmic suspension composition comprising of brinzolamide or a salt thereof and latanoprost or its salt thereof, stabilizer along with one or more pharmaceutically acceptable excipients for the treatment of elevated intraocular pressure (IOP) in patients with ocular hypertension or open-angle glaucoma. The present invention also provides a method of treating elevated intraocular pressure (IOP) in patients with ocular hypertension or open-angle glaucoma by administering an ophthalmic suspension composition comprising of brinzolamide or a salt thereof and latanoprost or a salt thereof, stabilizer and one or more pharmaceutically acceptable excipients.

### FIELD OF THE INVENTION

The present invention is related to ophthalmic suspension composition comprising of brinzolamide or a salt thereof, latanoprost or a salt thereof, stabilizer and one or more pharmaceutically acceptable excipients. The composition is useful for treatment of elevated intraocular pressure (IOP) in patients with ocular hypertension or open-angle glaucoma. The present invention is further related to a method of treating elevated intraocular pressure (IOP) in patients with ocular hypertension or open-angle glaucoma by administering an ophthalmic suspension composition comprising of brinzolamide or a salt thereof and latanoprost or a salt thereof, stabilizer and one or more pharmaceutically acceptable excipients.

### BACKGROUND OF THE INVENTION

Glaucoma is a disease with characteristic optic nerve damage and a corresponding visual field defect. Increased intraocular pressure (IOP) is one of the main risk factors. However, disturbances in blood flow may also play a role in some cases. In ocular hypertension, patients may have increased IOP but without changes in the visual field or corresponding optic nerve damage. Commonly prescribed drugs for treatment of elevated IOP in patients with ocular hypertension or open-angle glaucoma include prostaglandins, carbonic anhydrase inhibitors, beta blockers, alpha-adrenergic agonists, Rho kinase inhibitors and miotic/cholinergic agents.

Prostaglandins increase the outflow of the fluid through the eye thereby reducing the eye pressure. Medicines in this category include latanoprost (Xalatan™), travoprost (Travatan Z™), tafluprost (Zioptan™), bimatoprost (Lumigan™) and latanoprostene bunod (Vyzulta™).

Carbonic anhydrase inhibitors reduce the production of fluid in the eye. Examples include dorzolamide (Trusopt™) and brinzolamide (Azopt™). This class of medicine is usually prescribed to be used three times a day.

Beta blockers reduce the production of fluid in the eye, thereby lowering the pressure in the eye. Examples include timolol (Betimol™, Istalol™, Timoptic™) and betaxolol (Betoptic™).

Alpha-adrenergic agonists reduce the production of aqueous humor and increase outflow of the fluid through the eye. Examples include apraclonidine (Iopidine™) and brimonidine (Alphagan P™, Qoliana™). Alphagan P™ and Iopidine™ are meant for three times daily administration. Rho kinase inhibitors lower eye pressure by suppressing the rho kinase enzymes responsible for fluid increase. Examples include netarsudil (Rhopressa™) and is prescribed for once-a-day use. Possible side effects include eye redness, eye discomfort and deposits forming on the cornea.

Miotic or cholinergic agents increase the outflow of fluid from the eye. An example is pilocarpine (Isopto Carpine™). Side effects include headache, eye ache, smaller pupils, possible blurred or dim vision, and nearsightedness. This class of medicine is usually prescribed to be used up to four times a day.

Because of potential side effects and the need for frequent daily use, these medications lead to poor medication adherence in patients.

However, to treat the large population of glaucoma, physicians advise moving in a stepwise fashion, trying one method at a time and then supplementing with others in cases of unimproved symptoms by monotherapies or due to higher severity of disease. Further, there are concerns and expressed reservations in the ophthalmic community about patient compliance when the patient is required to administer separate medications to treat a single disease or condition such as glaucoma.

Further, when two medications are instilled too close in time, the application of the second medication may reduce the effect of the first drug. To overcome these potential problems, fixed combination (FC) therapy, in which two different drugs are mixed and instilled together in the same drop is required.

U.S. Pat. No. 11,285,163 discloses brinzolamide and latanoprost composition which is in the form of solution and brinzolamide is solubilized using Soluplus®. However, inherent disadvantages of such dosage form are their inability to stay for longer period of time with high local concentration, thus leading to poor bioavailability (BA).

Latanoprost is a practically insoluble prostaglandin F2α analog. From a pharmaceutical point of view, latanoprost is challenging to be formulated as an eye drop due to its poor water solubility and the presence of an ester bond that needs to be cleaved in vivo but maintained unchanged during storage. Hence, developing a formulation of latanoprost with good stability is a major challenge.

Therefore, there exists a long felt need for a topical ophthalmic pharmaceutical composition which is stable, capable of treating increased IOP along with reduced administration frequency. Unexpectedly it has been discovered that the suspension composition comprising brinzolamide, latanoprost and stabilizer meets these criteria of stability, efficacy and provides increased patient compliance.

### SUMMARY OF INVENTION

An aspect the present invention provides an ophthalmic suspension composition comprising of brinzolamide or a salt thereof and latanoprost or a salt thereof, stabilizer and one or more pharmaceutically acceptable excipients.

Another aspect of the present invention provides an ophthalmic suspension composition comprising of brinzolamide or a salt thereof and latanoprost or a salt thereof, stabilizer and one or more pharmaceutically acceptable excipients, wherein the composition is administered once daily.

Yet another aspect of the present invention provides an ophthalmic suspension composition comprising of brinzolamide or a salt thereof and latanoprost or a salt thereof, stabilizer and one or more pharmaceutically acceptable excipients, wherein the ratio of brinzolamide:latanoprost:stabilizer is 200:1:40-125.

Yet another aspect of the present invention provides an ophthalmic suspension composition comprising of brinzolamide or a salt thereof and latanoprost or a salt thereof, stabilizer and one or more pharmaceutically acceptable excipients, wherein the ratio of brinzolamide:latanoprost:stabilizer is 200:1:40-80.

Yet another aspect of the present invention provides an ophthalmic suspension composition comprising of brinzolamide or a salt thereof and latanoprost or a salt thereof, stabilizer and one or more pharmaceutically acceptable excipients, wherein the ratio of brinzolamide:latanoprost:stabilizer is 200:1:40-60.

Yet another aspect of the present invention provides an ophthalmic suspension composition comprising of brinzolamide or a salt thereof and latanoprost or a salt thereof, stabilizer and one or more pharmaceutically acceptable excipients, wherein the amount of stabilizer is 0.1 to 1.0% w/v of the composition.

Yet another aspect of the present invention provides an ophthalmic suspension composition comprising of brinzolamide or a salt thereof and latanoprost or a salt thereof, stabilizer and one or more pharmaceutically acceptable excipients, wherein the pharmaceutically acceptable excipients are selected from the group comprising buffering agent, preservative, chelating agent, pH-adjusting agent, tonicity-adjusting agent, penetration enhancer, thickener/viscosity modifier/gelling agent, demulcents/humectant, polyol, polymers, solvent/co-solvent/vehicle and/or combinations thereof.

Yet another aspect of the present invention provides an ophthalmic suspension composition, wherein the stabilizer is selected from the group comprising of cyclodextrin or its derivative, tyloxapol, polysorbate, Polyoxyethylene hydrogenated castor oil (macrogolglycerol hydroxystearate), polyoxyl 15 hydroxystearate or macrogol 15 hydroxystearate, Poly (ethyleneoxide)/poly(propyleneoxide) triblock copolymers (Pluronics®/Poloxamers®), polyvinyl caprolactam-polyvinyl acetate-polyethylene glycol graft co-polymer, polyoxyethylene fatty acid esters, ethoxylated alkyl phenol (e.g. Octoxynol-40), polyoxyethylene alkylphenyl ethers, and polyoxyethylene alkyl ethers and/or combinations thereof.

Yet another aspect of the present invention provides an ophthalmic suspension composition, wherein, the stabilizer is selected from the group comprising cyclodextrin or its derivative, or polysorbate and/or combinations thereof.

Yet another aspect of the present invention provides an ophthalmic suspension composition, wherein the buffering agent is selected from the group comprising bicarbonate buffer, acetate buffer, citrate buffer, phosphate buffer (sodium phosphate monohydrate, sodium phosphate dihydrate), borate buffer, diethanolamine, triethanolamine or tromethamine (TRIS, 2-amino-2-hydroxymethyl-1,3-propanediol) buffer, histidine buffer, and/or combinations thereof. In another aspect, the buffering agent is selected from the group comprising of sodium phosphate monohydrate, sodium phosphate dihydrate, citric acid, sodium citrate and/or combinations thereof.

Yet another aspect of the present invention provides an ophthalmic suspension composition comprising of brinzolamide or a salt thereof and latanoprost or a salt thereof, stabilizer and one or more pharmaceutically acceptable excipients, wherein the composition when stored for 6 months at 25 °C±2 °C/60%±5% RH, exhibits not more than 1-3% of total impurities.

Yet another aspect of the present invention provides an ophthalmic suspension composition comprising of brinzolamide or a salt thereof and latanoprost or a salt thereof, stabilizer and one or more pharmaceutically acceptable excipients, wherein the composition when stored for 1 month at 25 °C±2 °C/60%±5% RH, exhibits not more than 1% of total impurities.

Yet another aspect of the present invention provides an ophthalmic suspension composition comprising of brinzolamide or a salt thereof and latanoprost or a salt thereof, stabilizer and one or more pharmaceutically acceptable excipients, wherein the composition when stored for 6 months at 25 °C±2 °C/60%±5% RH, exhibits not more than 2.5% latanoprost acid, preferably not more than 2% latanoprost acid and most preferably not more than 1.5% latanoprost acid.

Yet another aspect of the present invention provides an ophthalmic suspension composition comprising of brinzolamide or a salt thereof and latanoprost or a salt thereof, stabilizer and one or more pharmaceutically acceptable excipients, wherein the composition, when stored for 1 month at 25 °C±2 °C/60%±5% RH, exhibits not more than 1% latanoprost acid.

Yet another aspect of the present invention provides an ophthalmic suspension composition comprising of brinzolamide or a salt thereof and latanoprost or a salt thereof, stabilizer and one or more pharmaceutically acceptable excipients, wherein the composition is preservative free.

Yet another aspect of the present invention provides an ophthalmic composition comprising of brinzolamide or a salt thereof and latanoprost or a salt thereof, stabilizer and one or more pharmaceutically acceptable excipients, wherein the composition provides synergistic effect as compared to individual drugs when administered separately.

Yet another aspect of the present invention provides an ophthalmic composition comprising of brinzolamide or a salt thereof and latanoprost or a salt thereof, stabilizer and one or more pharmaceutically acceptable excipients, wherein the composition leads to earlier drop in intraocular pressure after a specified period of treatment as compared to individual drugs when administered separately.

Yet another aspect of the present invention provides an ophthalmic composition comprising of brinzolamide or a salt thereof and latanoprost or a salt thereof, stabilizer and one or more pharmaceutically acceptable excipients, wherein the composition leads to reduced side effects as compared to individual drugs when administered separately.

Yet another aspect of the present invention provides an ophthalmic composition comprising of brinzolamide or a salt thereof and latanoprost or a salt thereof, stabilizer and one or more pharmaceutically acceptable excipients, wherein the composition leads to prolonged ocular residence time of the drug as compared to individual drugs when administered separately.

Yet another aspect of the present invention provides an ophthalmic suspension composition comprising of brinzolamide or a salt thereof and latanoprost or a salt thereof, stabilizer and one or more pharmaceutically acceptable excipients, wherein the composition results in enhanced ocular bioavailability as compared to individual drugs when administered separately.

Yet another aspect of the present invention provides an ophthalmic suspension composition for treatment of elevated intraocular pressure (IOP) in patients with ocular hypertension or open-angle glaucoma, wherein the composition comprising of brinzolamide or a salt thereof and latanoprost or a salt thereof, stabilizer and one or more pharmaceutically acceptable excipients.

Yet another aspect of the present invention provides a method of treating elevated intraocular pressure (IOP) in patients with ocular hypertension or open-angle glaucoma by administering an ophthalmic suspension composition comprising of brinzolamide or a salt thereof and latanoprost or a salt thereof, stabilizer and one or more pharmaceutically acceptable excipients.

### DESCRIPTION OF THE INVENTION

At the very outset of the detailed description, it can be understood that the ensuing description only illustrates a particular form of this invention. However, such a particular form is an only exemplary embodiment, and without intending to imply any limitation on the scope of this invention.

Accordingly, the description is understood as an exemplary embodiment and teaching of invention and is not intended to be taken restrictively.

Unless defined otherwise, technical and scientific terms used herein have the same meaning as commonly understood by person of ordinary skill in the art to which this invention belongs. Further, unless otherwise required by context, singular terms shall include pluralities, and plural terms shall include the singular.

It is to be understood that the present disclosure is not limited in its application to the details of the method set forth in the following description. The present disclosure is capable of other embodiments and of being practiced or of being carried out in various ways. Also, it is to be understood that the phraseology and terminology used herein is for the purpose of description and should not be regarded as limiting.

Each embodiment is provided by way of explanation of the invention and not by way of limitation of the invention. In fact, it will be apparent to a person skilled in the art that various modifications and variations can be made to the methods described herein without departing from the scope or spirit of the invention. For instance, features illustrated or described as part of one embodiment can be applied to another embodiment to yield a still further embodiment. Therefore, it is intended that the present invention includes such modifications and variations and their equivalents. Other objects, features, and aspects of the present disclosure are disclosed in or are obvious from, the following detailed description. It is to be understood by a person skilled in the art that the present discussion is a description of exemplary embodiments only and is not to be construed as limiting the broader aspects of the present invention.

Main embodiment of the present invention provides an ophthalmic suspension composition comprising of brinzolamide or a salt thereof and latanoprost or its salt thereof.

An embodiment of the present invention provides an ophthalmic suspension composition comprising:
brinzolamide or a salt thereof;
latanoprost or a salt thereof;
A stabilizer; and
one or more pharmaceutically acceptable excipients.

Another embodiment of the present invention provides an ophthalmic suspension composition comprising of brinzolamide or a salt thereof and latanoprost or a salt thereof, stabilizer and one or more pharmaceutically acceptable excipients, wherein the composition is administered once daily.

Brinzolamide is a carbonic anhydrase II (CA-II) inhibitor, chemically known as (R)-(+)-4-Ethylamino-2-(3-methoxypropyl)-3,4-dihydro-2H-thieno[3,2-e]-1,2-thiazine-6-sulfonamide-1,1-dioxide. Its empirical formula is C₁₂H₂₁N₃O₅S₃. Brinzolamide has a molecular weight of 383.5 g/mol and a melting point of about 131 °C. It is a white powder, which is insoluble in water, very soluble in methanol and soluble in ethanol.

Latanoprost is a prostaglandin F2a analogue, chemically known as isopropyl-(Z)-7[(1R,2R,3R,5S) 3,5 dihydroxy-2-[(3R)-3-hydroxy-5-phenylpentyl]cyclopentyl]-5-heptenoate. Its molecular formula is C₂₆H₄₀O₅. Latanoprost is a colorless to slightly yellow oil that is very soluble in acetonitrile and freely soluble in acetone, ethanol, ethyl acetate, isopropanol, methanol, and octanol. It is practically insoluble in water.

Yet another embodiment of the present invention provides an ophthalmic composition comprising of brinzolamide or a salt thereof and latanoprost or its salt thereof.

Yet another embodiment of the present invention provides an ophthalmic composition comprising brinzolamide or a salt thereof and latanoprost or a salt thereof, stabilizer and one or more pharmaceutically acceptable excipients.

Yet another embodiment of the present invention provides the composition, wherein the composition is administered once daily.

Yet another embodiment of the present invention provides the composition, wherein the ratio of brinzolamide or a salt thereof:latanoprost or a salt thereof:stabilizer is 200:1:40-125.

Yet another embodiment of the present invention provides the composition, wherein the ratio of brinzolamide or a salt thereof:latanoprost or a salt thereof:stabilizer is 200:1:40-80.

Yet another embodiment of the present invention provides the composition, wherein the ratio of brinzolamide or a salt thereof:latanoprost or a salt thereof:stabilizer is 200:1:40-60.

Yet another embodiment of the present invention provides the composition, wherein the composition contains brinzolamide or a salt thereof in an amount of about 0.01% w/v to about 10% w/v of the composition.

Yet another embodiment of the present invention provides the composition, wherein the composition contains latanoprost or a salt thereof in an amount of about 0.01% w/v to about 5% w/v of the composition.

Yet another embodiment of the present invention provides the composition, wherein the composition may comprise the amorphous form of active ingredient or any of its crystalline form or a combination thereof.

Stabilizer is selected from the group comprising cyclodextrin (e.g. HPβCD, HPγCD); cyclodextrin derivatives such as ether and mixed ether derivatives and those derivatives bearing sugar residues, tyloxapol, polysorbate, Polyoxyethylene hydrogenated castor oil (macrogolglycerol hydroxystearate), polyoxyl 15 hydroxystearate or macrogol 15 hydroxystearate, Poly (ethyleneoxide)/poly(propyleneoxide) triblock copolymers (Pluronics®/Poloxamers®), polyvinyl caprolactam-polyvinyl acetate-polyethylene glycol graft co-polymer, polyoxyethylene fatty acid esters, ethoxylated alkyl phenol (e.g. Octoxynol-40), polyoxyethylene alkylphenyl ethers, and polyoxyethylene alkyl ethers and/or combinations thereof.

Yet another embodiment of the present invention provides the composition, wherein the stabilizer is selected from the group comprising cyclodextrin or its derivative, polysorbate and/or combinations thereof.

Stabilizer is present in an amount of about 0.001% w/v to about 10.0% w/v by weight of the composition. Preferably, stabilizer is present in the amount of 0.1% to 1% w/v by weight of the composition.

Yet another embodiment of the present invention provides the composition, wherein the ophthalmic composition may also contain other pharmaceutically acceptable ingredients such as one or more pharmaceutically acceptable buffering agent, preservative, chelating agent, pH-adjusting agent, tonicity-adjusting agent, penetration enhancer, thickener/viscosity modifier/gelling agent, demulcents/humectant, polyol, polymers, solvent/co-solvent/vehicle and/or combinations thereof.

Suitable buffering agent is selected from the group comprising bicarbonate buffer, acetate buffer, citrate buffer, phosphate buffer (sodium phosphate monohydrate, sodium phosphate dihydrate), borate buffer, diethanolamine, triethanolamine or tromethamine (TRIS, 2-amino-2-hydroxymethyl-1,3-propanediol) buffer, histidine buffer, and/or combinations thereof. Buffering agent is present in an amount of about 0.001% w/v to about 10.0% w/v by weight of the composition.

Suitable preservative is selected from the group comprising benzalkonium chloride, benzethonium chloride, benzododecinium chloride, chlorobutanol, chlorhexidine, parabens, stabilized oxychloro compounds, purite, benzyl alcohol, phenylmercuric acetate, phenylmercuric nitrate, thimerosal, sorbic acid salts such as potassium sorbate, calcium sorbate, sodium sorbate, polyaminopropyl biguanide, pyruvates, polyquaternium-1, polyhexamethylene biguanide (PHMB), PVP-Iodine complex, metal ions, peroxides, aminoacids and/or combinations thereof. Preservative is present in an amount of about 0.001% w/v to about 10.0% w/v by weight of the composition.

Yet another embodiment of the present invention provides the composition, wherein the ophthalmic composition comprising brinzolamide or a salt thereof and latanoprost or a salt thereof, wherein the composition is preservative free.

Suitable chelating agent is selected from the group comprising edetate disodium, ethylenediamine tetracetic acid (EDTA), edetic acid, disodium edetate dihydrate, diethylenetriamine pentaacetic acid, citric acid, malic acid, phytic acid, pyrophasphoric acid, alkali metal hexametaphosphate and/or combinations thereof. Other acceptable chelating agents such as amino acids, like glutamic and aspartic acids and borates may be used. Chelating agent is present in an amount of about 0.001% w/v to about 10.0% w/v by weight of the composition.

Suitable pH adjusting agent is selected from the group comprising hydrochloric acid, phosphoric acid, acetic acid, boric acid, citric acid, tartaric acid, sodium hydroxide, potassium hydroxide, sodium carbonate, sodium hydrogen carbonate, tromethamine, arginine, lysine, histidine, guanine or salts thereof and/or combinations thereof.

Suitable tonicity adjusting agent is selected from the group comprising alkali metal chlorides and alkaline earth metal chlorides, such as sodium chloride, potassium chloride, calcium chloride and magnesium chloride, sodium nitrate, sodium sulfate, sodium bromide, sodium phosphate, glycerol, propylene glycol, polyethylene glycols (PEG), dextrose and/or combinations thereof. Tonicity agent is present in an amount of about 0.001% w/v to about 10.0% w/v by weight of the composition.

Suitable penetration enhancer is selected from the group comprising dimethylsulphoxide (DMSO), ethylenediaminetetraacetic acid (EDTA), sodium glycocholate & related cholates, saponins, bile salts and/or combinations thereof. The present invention contains penetration enhancer in an amount of about 0.01% w/v to about 10.0% w/v of the composition.

Suitable thickening agent is selected from the group comprising hydroxyalkylated cellulose, alkylated cellulose, sodium carboxymethyl cellulose, chondroitin sulfate, polyacrylic acid, polyvinyl alcohol, polyethylene glycol, polysaccharides, polyvinylpyrrolidone, hyaluronic acid, carboxyvinyl polymer (Carbomer 974 P), xanthan gum, gellan gum, alginic acid, carageenans and/or combinations thereof. The hydroxyalkylated cellulose may be hydroxypropylcellulose, hydroxymenthyl cellulose, hydroxyethylcellulose or hydroxypropylmethylcellulose. The alkylated cellulose may be methylcellulose or ethylcellulose. The present invention contains thickening agent in an amount of about 0.1% w/v to about 50.0% w/v of the composition. Suitable humectant is selected from the group comprising polyvinyl alcohol, polyvinyl pyrrolidone, cellulose derivatives, glycerin, sorbitol, mannitol, polyethylene glycol and/or combinations thereof. Humectant is present in an amount of about 0.001% w/v to about 10.0% w/v of the composition.

Suitable polyol is selected from the group comprising mannitol, sorbitol, propylene glycol, glycerin, maltitol, lactitol, xylitol, isomalt, erythritol, dextrose and/or combinations thereof. Polyol is present in an amount of about 0.001% w/v to about 10.0% w/v of the composition.

Suitable polymers, include, but are not limited to, natural and synthetic polymers, polysaccharides, polyaminoglycosides, cellulose derivatives, acrylic acid derivatives, guar gum, xanthan gum, geltrite, dextran, hyaluronate, chondroitin sulfate, locust bean gum, polyvinyl alcohol, polyvinyl pyrrolidone, carbopol, polycarbophil, polystyrene sulfonate and/or combinations thereof. The present invention contains polymer in an amount of about 0.1% w/v to about 50.0% w/v by weight of the composition.

Yet another embodiment of the present invention provides the composition, wherein the composition comprising of brinzolamide or a salt thereof and latanoprost or a salt thereof, stabilizer and one or more pharmaceutically acceptable excipients, wherein the composition is free of Soluplus® Suitable solvents/co-solvents may be selected from a group comprising water for injection, normal saline solution, Ringer's solution, alcohols, such as ethanol, and glycols such as ethylene glycol, propylene glycol, polyethylene glycol, glycofurol, glycerin, polyvinyl alcohol, povidone, glycosaminoglycans such as sodium hyaluronate and/or combinations thereof.

Yet another embodiment of the present invention provides the composition, wherein the ophthalmic composition is provided/supplied in a single dose container or a multi-dose container.

The term “multi-dose” as used herein is defined as a composition wherein the composition can be administered multiple times from the same container.

The term “container” refers to vials, ampoules, bottles, UNIMS, pre-filled syringes etc.

Yet another embodiment of the present invention provides the composition, wherein the ophthalmic composition comprising brinzolamide or a salt thereof and latanoprost or a salt thereof, wherein the composition is in the form of solution, suspension, emulsion, gel, gel forming solution, drops, implant, insert, ointment or dispersion thereof. Preferably, the composition is in the form of suspension.

Particle size in suspensions for ocular drug delivery is important. An increase in drug particle size enhances the ocular bioavailability. Unfortunately, it may result in a foreign body sensation in the eye following ocular application, causing reflex tearing. On the other hand, reduction in particle size generally improves the patient comfort and acceptability of suspension formulations but it may lead to changes in particle size due to Ostwald ripening or particle agglomeration. Therefore, particle size needs to be evaluated through stability testing.

D10 represents the 10% of total particles are smaller than this size. D90 means that 90% of the total particles are smaller than this size. D50 means that 50% of the total particles are smaller than this size. SPAN is expressed by the equation SPAN=(D90−D10)/D50 wherein D90, D10 and D50 are particle sizes at 90%, 10% and 50% in a volume cumulative distribution, respectively, while D50 is also referred to as the median diameter. The smaller the value is, the narrower the particle size distribution is.

Yet another embodiment of the present invention provides an ophthalmic composition comprising brinzolamide or a salt thereof and latanoprost or a salt thereof, wherein the suspension has particle size distribution of D50 in the range of 0.5-1 μm and D90 in the range of 1-2.5 μm.

Yet another embodiment of the present invention provides an ophthalmic composition comprising brinzolamide or a salt thereof and latanoprost or a salt thereof, wherein the suspension has particle size distribution having SPAN value of less than 2.5, preferably in the range of 1 to 2.

Yet another embodiment of the present invention provides the use of high pressure homogenizer, microfluidizer, ball milling, bead milling or jet milling to reduce the particle size. Preferably, bead milling is used for reduction of particle size.

Yet another embodiment of the present invention provides the ophthalmic composition which has pH in the range of about 5.0 to about 8.0.

Yet another embodiment of the present invention provides the ophthalmic composition which has viscosity in the range of 5 to 100 cps.

Yet another embodiment of the present invention provides the ophthalmic suspension composition comprising of brinzolamide or a salt thereof and latanoprost or a salt thereof, stabilizer and one or more pharmaceutically acceptable excipients, wherein the composition is stable when subjected to stress studies and stability studies under long-term and accelerated storage conditions.

Yet another embodiment of the present invention provides the ophthalmic suspension composition comprising of brinzolamide or a salt thereof and latanoprost or a salt thereof, stabilizer and one or more pharmaceutically acceptable excipients, wherein the composition, when stored for 6 months at 25 °C±2 °C/60%±5% RH, exhibits not more than 1-5% of total impurities, preferably not more than 1-3% of total impurities.

Yet another embodiment of the present invention provides the ophthalmic suspension composition comprising of brinzolamide or a salt thereof and latanoprost or a salt thereof, stabilizer and one or more pharmaceutically acceptable excipients, wherein the composition when stored for 6 months at 25 °C±2 °C/60%±5% RH, exhibits not more than 1.5% latanoprost acid.

Yet another embodiment of the present invention provides the ophthalmic suspension composition comprising of brinzolamide or a salt thereof and latanoprost or a salt thereof, stabilizer and one or more pharmaceutically acceptable excipients, wherein the composition, when stored for 1 month at 25 °C±2 °C/60%±5% RH, exhibits not more than 1% of total impurities.

Yet another embodiment of the present invention provides the ophthalmic suspension composition comprising of brinzolamide or a salt thereof and latanoprost or a salt thereof, stabilizer and one or more pharmaceutically acceptable excipients, wherein the composition, when stored for 6 months at 25 °C±2 °C/60%±5% RH, exhibits not more than 2.5% latanoprost acid, preferably not more than 2% latanoprost acid and most preferably not more than 1.5% latanoprost acid.

Yet another embodiment of the present invention provides the ophthalmic suspension composition comprising of brinzolamide or a salt thereof and latanoprost or a salt thereof, stabilizer and one or more pharmaceutically acceptable excipients, wherein the composition, when stored for 1 month at 25 °C±2 °C/60%±5% RH, exhibits not more than 1% latanoprost acid.

Yet another embodiment of the present invention provides the ophthalmic suspension composition comprising of brinzolamide or a salt thereof and latanoprost or a salt thereof, stabilizer and one or more pharmaceutically acceptable excipients, wherein the composition, when stored for 6 months at 2 °C-8 °C, exhibits not more than 1-3% of total impurities, preferably not more than 1% of total impurities.

Yet another embodiment of the present invention provides the ophthalmic composition which provides synergistic effect as compared to individual drugs when administered separately.

Yet another embodiment of the present invention provides the ophthalmic composition which leads to earlier drop in intraocular pressure after a specified period of treatment as compared to individual drugs when administered separately.

Yet another embodiment of the present invention provides the ophthalmic composition which leads to the reduction in intraocular pressure for a duration of at least 3-24 hours. Yet another embodiment of the present invention provides the ophthalmic composition which leads to reduced side effects as compared to individual drugs when administered separately.

Yet another embodiment of the present invention provides the ophthalmic composition which leads to prolonged ocular residence time of the drug. As a suspension, API particles tend to stay in the eyes and dissolve gradually over a longer period of time, thereby resulting in enhanced ocular bioavailability.

Yet another embodiment of the present invention provides the ophthalmic suspension composition comprising of brinzolamide or a salt thereof and latanoprost or a salt thereof, stabilizer and one or more pharmaceutically acceptable excipients, wherein the composition results in enhanced ocular bioavailability as compared to individual drugs when administered separately.

Yet another embodiment of the present invention provides an ophthalmic composition comprising of brinzolamide or a salt thereof and latanoprost or a salt thereof, stabilizer and one or more pharmaceutically acceptable excipients, wherein the composition leads to prolonged ocular residence time of the drug as compared to individual drugs when administered separately.

Yet another embodiment of the present invention provides an ophthalmic suspension composition for treatment of elevated intraocular pressure (IOP) in patients with ocular hypertension or open-angle glaucoma, wherein the composition comprising of brinzolamide or a salt thereof and latanoprost or a salt thereof, stabilizer and one or more pharmaceutically acceptable excipients.

Yet another embodiment of the present invention provides the pharmaceutical composition which is meant to be administered once daily or several times per day depending upon the need of the patient.

Yet another embodiment of the present invention provides the pharmaceutical composition which can be sterilized using any of the known methods of sterilization, such as filtration, moist heat/autoclaving, dry heat, gas sterilization or irradiation (gamma and electron beam).

Yet another embodiment of the present invention provides the pharmaceutical composition which is stable when subjected to stability testing conditions.

Yet another embodiment of the present invention provides the composition which contains reduced total impurities when the composition contains stabilizer as compared to the composition which does not contain stabilizer.

Yet another embodiment of the present invention provides the ophthalmic composition which has an osmolality of about 100 mOsm/kg to about 500 mOsm/kg, preferably in the range of 200 to about 400 mOsm/kg.

Rheology is another critical quality attribute that impacts on the physical stability, spreadability, ease of application, drug release and ocular residence time of an ophthalmic suspension. Ideally, a non-solution eye drop formulation should have sufficient viscosity to prolong ocular retention and at the same time readily flowable under shear (e.g., after administration or during blinking) to ensure uniform coverage of the eye surface.

Yet another embodiment of the present invention provides the ophthalmic composition which has a viscosity of between 5 and 300 cps.

Yet another embodiment of the present invention provides the ophthalmic composition which can be stored in a plastic container or glass container. The material of the plastic container comprises high density polyethylene (HDPE), low density polyethylene (LDPE), polyethylene terephthalate (PET), polyvinyl chloride (PVC), polypropylene (PP), polystyrene (PS), and/or combination thereof.

Yet another embodiment of the present invention provides a method of treating elevated intraocular pressure (IOP) in patients with ocular hypertension or open-angle glaucoma by administering an ophthalmic suspension composition comprising of brinzolamide or a salt thereof and latanoprost or a salt thereof, stabilizer and one or more pharmaceutically acceptable excipients.

While the present invention is disclosed generally above, additional aspects are further discussed and illustrated with reference to the examples below. However, the examples are presented merely to illustrate the invention and should not be considered as limitations thereto.

### Example 1

| Ingredients | Quantity (mg/ml) |
|---|---|
| Latanoprost | 0.05 |
| Brinzolamide | 10.00 |
| Benzalkonium Chloride | 0.20 |
| Sodium chloride | 2.50 |
| Edetate disodium | 0.10 |
| Carbomer | 4.50 |
| Mannitol | 33.00 |
| Tyloxapol | 0.25 |
| Sodium hydroxide | q.s. to adjust pH |
| Hydrochloric acid | q.s. to adjust pH |
| Water for injection | q.s. to 100% |

### Method of Preparation:

### Preparation of Polymer Phase:

1) Taking 50% (of actual batch size) of water for injection in a container.
2) Adding the dispensed quantity of edetate disodium into the above water for injection of step 1 under continuous stirring to get a clear solution.
3) Adding the dispensed quantity of mannitol into the solution of step 2 under continuous stirring (rpm 700±200) to get a clear solution.
4) Adding the dispensed quantity of carbomer into the above solution of step 3 to form homogeneous solution.
5) Adding the dispensed quantity of sodium chloride to the above solution of step 4 under continuous stirring to get it dissolved completely.
6) Checking the pH of above solution of step 5 and if required adjusting the pH between 6.0 to 7.0 with sodium hydroxide solution.
7) Stirring for 15 minutes and making the volume up to 60% of the standard batch size.
8) Filtering the above solution of step 7 through 20 μm filter.
9) Autoclaving the above solution of step 8 at 121 °C for 30 minutes and then cooling it at room temperature to form polymer phase.

### Preparation of Latanoprost Phase:

10) Collecting 2% (of actual batch size) of hot water for injection water in a clean container.
11) Adding dispensed quantity of benzalkonium chloride into water for injection of step 10.
12) Adding latanoprost to solution of step 11.
13) Sonicating the solution of step 12 for 30 minutes at 50 °C-60 °C

### Preparation of Polymer Phase Containing Latanoprost

14) Adding the solution of step 13 into polymer phase of step 9 under continuous stirring to form polymer phase containing latanoprost.

### Preparation of Brinzolamide and Tyloxapol Slurry:

15) Taking 5% (of actual batch size) of hot water for injection.
16) Dissolving weighed quantity of tyloxapol into water for injection of step 15.
17) Filtering the solution of step 16 using 0.2 μm filter.
18) Adding dispensed quantity of brinzolamide into the above filtered tyloxapol solution of step 17 under continuous stirring to form slurry.
19) Milling the slurry obtained in step 18 by using bead mill to get desired particle size.

### Bulk Preparation

20) Adding the milled slurry of step 19 to the polymer phase containing latanoprost obtained in step 14 under continuous stirring.
21) Making up the volume up to 100% with water for injection.
22) Filling the final suspension obtained in step 21 in suitable container.

### Example 2

| Ingredients | Quantity (mg/ml) |
|---|---|
| Latanoprost | 0.05 |
| Brinzolamide | 10.00 |
| Benzalkonium chloride | 0.20 |
| Edetate disodium | 0.10 |
| Carbomer | 4.20 |
| Mannitol | 33.00 |
| Tyloxapol | 0.25 |
| Hydroxypropylbetadex | 2.4-5.4 |
| Sodium hydroxide | q.s. |
| Hydrochloric acid | q.s. |
| Sodium Chloride | 2.50 |
| Milli Q water | q.s. |

### Process:

### Preparation of Polymer Phase:

1) Taking 40% (of actual batch size) of water for injection in a container.
2) Adding the dispensed quantity of Carbomer under stirring. Stir till get lumps free solution. Heat the solution at 40-45 °C under stirring. Cool the solution at room temperature. Adjust the pH of carbomer up to 7.3±0.2 with IN sodium hydroxide solution.
3) Collect 15% (of actual batch size) of water, add edetate disodium into the above water for injection under continuous stirring to get a clear solution.
4) Adding the dispensed quantity of mannitol into the solution of step 3 under continuous stirring (rpm 700±200) to get a clear solution.
5) Adding the dispensed quantity of sodium chloride into the above solution of step 4 under continuous stirring (rpm 700±200) to get a clear solution.
6) Adding the dispensed quantity of benzalkonium chloride to above solution of step 5 under continuous stirring (rpm 700±200) to get a clear solution.
7) Add the step 6 solution to step 2 under stirring and mix well. Checking the pH of the solution and if required adjusting the pH between 7.3±0.2 with sodium hydroxide solution.
8) Stirring for 15 minutes and making the volume up to 60% of the standard batch size.
9) Filtering the above solution of step 8 through 20 μm filter.
10) Autoclaving the above solution of step 9 at 121 °C for 30 minutes and then cooling it at room temperature to form polymer phase.

### Preparation of Latanoprost Phase:

11) Collecting 1% (of actual batch size) of water for injection water in a clean container.
12) Adding dispensed quantity of Hydroxypropylbetadex into water for injection of step 11.
13) Adding latanoprost to solution of step 12.
14) Stir the solution till latanoprost dissolve completely.

### Preparation of Polymer Phase Containing Latanoprost

15) Adding the solution of step 14 into polymer phase of step 10 under continuous stirring to form polymer phase containing latanoprost.

### Preparation of Brinzolamide and Tyloxapol Slurry:

16) Taking 5% (of actual batch size) of hot water for injection.
17) Dissolving weighed quantity of tyloxapol into water for injection of step 16.
18) Filtering the solution of step 17 using 0.2 μm filter.
19) Adding dispensed quantity of brinzolamide into the above filtered tyloxapol solution of step 18 under continuous stirring to form slurry.
20) Milling the slurry obtained in step 19 by using bead mill to get desired particle size.

### Bulk Preparation

21) Adding the milled slurry of step 20 to the polymer phase containing latanoprost obtained in step 15 under continuous stirring.
22) Making up the volume up to 100% with water for injection.
23) Filling the final suspension obtained in step 21 in suitable container.

### Physical Parameters

The composition was prepared according to Example 1 and its physical parameters are presented in Table 1.

TABLE 1

| Physical parameters | Observations |
|---|---|
| pH | 7.29 |
| Osmolality (mOsm/kg) | 292 |
| Viscosity (cps) | 197 |

### Example 3

| Ingredients | Quantity (mg/ml) |
|---|---|
| Latanoprost | 0.05 |
| Brinzolamide | 10.00 |
| Benzalkonium chloride | 0.20 |
| Edetate disodium | 0.10 |
| Carbomer | 4.20 |
| Mannitol | 33.00 |
| Polysorbate | 0.20 |
| Hydroxypropyl betacyclodextrin | 2.40 |
| Sodium hydroxide | q.s. to adjust pH |
| Hydrochloric acid | q.s. to adjust pH |
| Sodium chloride | 2.50 |
| Milli Q water | q.s. |

### Process:

### Preparation of Polymer Phase:

1) Taking 40% (of actual batch size) of water for injection in a container.
2) Adding the dispensed quantity of Carbomer under stirring. Stir till get lumps free solution. Heat the solution at 40-45 °C under stirring. Cool the solution at room temperature. Adjust the pH of carbomer up to 7.3±0.2 with IN sodium hydroxide solution.
3) Collect 15% (of actual batch size) of water, add edetate disodium into the above water for injection under continuous stirring to get a clear solution.
4) Adding the dispensed quantity of mannitol into the solution of step 3 under continuous stirring (rpm 700±200) to get a clear solution.
5) Adding the dispensed quantity of sodium chloride into the above solution of step 4 under continuous stirring (rpm 700±200) to get a clear solution.
6) Adding the dispensed quantity of benzalkonium chloride to above solution of step 5 under continuous stirring (rpm 700±200) to get a clear solution.
7) Adding the solution of step 6 to step 2 under stirring and mix well. Checking the pH of the solution and if required adjusting the pH between 7.3±0.2 with sodium hydroxide solution.
8) Stirring for 15 minutes and making the volume up to 60% of the standard batch size.
9) Filtering the above solution of step 8 through 20 μm filter.
10) Autoclaving the above solution of step 9 at 121 °C for 30 minutes and then cooling it at room temperature to form polymer phase.

### Preparation of Latanoprost Phase:

11) Collecting 1% (of actual batch size) of water for injection water in a clean container.
12) Adding dispensed quantity of Hydroxypropylbetadex into water for injection of step 11.
13) Adding latanoprost to solution of step 12.
14) Stir the solution till latanoprost dissolve completely.

### Preparation of Polymer Phase Containing Latanoprost

15) Adding the solution of step 14 into polymer phase of step 10 under continuous stirring to form polymer phase containing latanoprost.

### Preparation of Brinzolamide and Polysorbate Slurry:

16) Taking 5% (of actual batch size) of hot water for injection.
17) Dissolving weighed quantity of polysorbate into water for injection of step 16.
18) Filtering the solution of step 17 using 0.2 μm filter.
19) Adding dispensed quantity of brinzolamide into the above filtered polysorbate solution of step 18 under continuous stirring to form slurry.
20) Milling the slurry obtained in step 19 by using bead mill to get desired particle size.

### Bulk Preparation

21) Adding the milled slurry of step 20 to the polymer phase containing latanoprost obtained in step 15 under continuous stirring.
22) Making up the volume up to 100% with water for injection.
23) Filling the final suspension obtained in step 21 in suitable container.

### Example 4

| Ingredients | Quantity (mg/ml) |
|---|---|
| Latanoprost | 0.05 |
| Brinzolamide | 10.00 |
| Benzalkonium chloride | 0.20 |
| Sodium Chloride | 4.10 |
| Polysorbate | 2.46 |
| Carbomer | 4.20 |
| Disodium hydrogen phosphate anhydrous | 4.74 |
| Sodium dihydrogen phosphate monohydrate | 4.60 |
| Sodium hydroxide | q.s. to pH |
| Hydrochloric acid | q.s. to pH |
| Water for Injection | q.s. to 1 mL |

### Process:

### Preparation of Carbomer Phase—

1. Collecting 60% of water for injection in a glass beaker.
2. Adding slowly Carbomer under continuous stirring to the water of injection of step 1.
3. Adding slowly Sodium chloride under continuous stirring to the solution obtained in step 2.
4. Adding slowly Sodium dihydrogen phosphate monohydrate under continuous stirring to solution of step 3.
5. Adding slowly Disodium hydrogen phosphate anhydrous under continuous stirring to solution of step 4.
6. Adjusting pH of Carbomer phase using sodium hydroxide solution to 7.3±0.2

### Preparation of Latanoprost Phase—

7. Collecting 2% (of actual batch size) of hot milli Q water (temp. 60-70 °C) in a clean glass beaker.
8. Adding Benzalkonium chloride into above beaker and dissolving it. Making up the volume with milli Q water to get 0.8% solution.
9. Dispensing Latanoprost in a glass bottle and adding 1/10 of benzalkonium chloride solution from step 8 solution into it. Sonicating it for 30 minutes at 50 °C-60 °C
10. Adding approx. half of the solution of latanoprost into polymer phase under continuous stirring.
11. Repeating the above step 9 & 10 for 6-7 times or till complete Latanoprost is transferred.

### Preparation of Brinzolamide Phase—

12. Taking 5% (of actual batch size) hot Milli Q water. Dissolving weighed quantity of polysorbate with hot Milli-Q water and stirring for 15 mins. Making the volume of the solution to 7.5% of the standard batch size and stirring for 15 mins. Filtering the solution with 0.2 μm filter and collecting it into a separate beaker.
13. Slowly adding dispensed quantity of Brinzolamide into above filtered polysorbate solution under continuous stirring. Stir for 60 minutes.
14. Milling the brinzolamide slurry by using bead mill to get desired particle size.

### Preparation of Bulk—

15. Adding the Brinzolamide milled slurry of step 14 to the carbomer phase containing Latanoprost of step 11 under continuous stirring.
16. Making up the volume up to 100% of the actual batch size with Milli Q water.
17. Filling the final suspension in previously sterilized low density polyethylene (LDPE) three piece bottles.

### Example 5

| Ingredients | Quantity (mg/ml) |
|---|---|
| Latanoprost | 0.05 |
| Brinzolamide | 10.00 |
| Benzalkonium chloride | 0.200 |
| PS80 | 2.46 |
| Glycerin | 19.76 |
| EDTA | 1.27 |
| Carbomer | 4.20 |
| Citric acid anhydrous | 0.88 |
| Sodium citrate dihydrate | 4.44 |
| Water for Injection | q.s. to 1 mL |

### Process

### Preparation of Carbomer Phase—

1. Collecting 60% water for injection in a glass beaker.
2. Adding slowly Carbopol under continuous stirring to water for injection of step 1.
3. Adding slowly EDTA under continuous stirring to solution of step 2.
4. Adding slowly Glycerin under continuous stirring to solution of step 3.
5. Adding slowly citric acid anhydrous under continuous stirring to solution of step 4.
6. Adding slowly Sodium citrate dihydrate under continuous stirring to solution of step 5.
7. Checking pH.
8. Autoclaving the above solution at 121 °C for 30 min.

### Preparation of Latanoprost Phase—

9. Collecting 2% (of actual batch size) of hot milli Q water (temp. 60-70 °C) in a clean glass beaker.
10. Adding slowly dispensed quantity of Benzalkonium chloride into above beaker and dissolving it. Making up the volume with milli Q water to get 0.8% solution.
11. Dispensing the Latanoprost in a glass bottle and adding 1/10 of benzalkonium chloride solution from above step 10 into it. Sonicating it for 30 minutes at 50 °C-60 °C.
12. Adding approximately half of the solution of latanoprost into polymer phase under continuous stirring.
13. Repeating the above step 11 & 12 for 6-7 times or till complete Latanoprost is transferred.

### Preparation of Brinzolamide Phase—

14. Taking 5% (of actual batch size) hot Milli Q water. Dissolving weighed quantity of polysorbate with hot Milli-Q water. Making the volume of the solution to 7.5% of the standard batch size and stirring for 15 mins. Filtering the solution with 0.2 μm filter and collecting it into a separate beaker.
15. Adding slowly dispensed quantity of Brinzolamide into above filtered polysorbate solution under continuous stirring.
16. Milling the brinzolamide slurry by using bead mill to get desired particle size.

### Preparation of Bulk—

17. Adding the Brinzolamide milled slurry of step 16 to the carbomer phase containing Latanoprost of step 13 under continuous stirring.
18. Making up the volume up to 100% of the actual batch size with Milli Q water.
19. Filling the final suspension in suitable container.

### Stress and Stability Data:

The composition prepared according to example 1-5 were subjected to stress studies at 40±2 °C/NMT 25% RH and Stability Studies at 25±2 °C/60±5% RH and 2 °C-8 °C. The results are summarized below in Table 2

TABLE 2

| | | | Highest | Other | |
|---|---|---|---|---|---|
| | | | unknown | unknown | |
| | | Latanoprost | impurities | impurities | Total Impurities |
| Condition | Example No. | Acid % | % | % | % |
| Initial | Example 1 | 75.63 | 1.89 | ND | 77.52 |
| | Example 2 | ND | ND | ND | ND |
| | Example 3 | ND | 0.14 | ND | 0.14 |
| | Example 4 | 0.09 | 0.55 | ND | 0.72 |
| | Example 5 | ND | ND | ND | BDL |
| 1W stress | Example 1 | NA | NA | NA | NA |
| (40 ± 2° C./NMT 25% | Example 2 | 0.33 | ND | ND | 0.33 |
| RH) | Example 3 | 0.21 | ND | ND | 0.21 |
| | Example 4 | 0.10 | 0.49 | ND | 0.59 |
| | Example 5 | 0.15 | ND | ND | 0.15 |
| 2W stress | Example 1 | NA | NA | NA | NA |
| (40 ± 2° C./NMT 25% | Example 2 | 0.46 | 0.27 | ND | 0.73 |
| RH) | Example 3 | 0.40 | ND | ND | 0.40 |
| | Example 4 | 1.26 | ND | ND | 1.26 |
| | Example 5 | 0.23 | ND | ND | 0.23 |
| 4W stress | Example 1 | NA | NA | NA | NA |
| (40 ± 2° C./NMT 25% | Example 2 | 1.92 | ND | ND | 1.92 |
| RH) | Example 3 | 0.60 | ND | ND | 0.60 |
| | Example 4 | 0.18 | ND | ND | 0.18 |
| | Example 5 | 0.53 | 0.08 | ND | 0.61 |
| 1M (25 ± 2° C./60 ± | Example 1 | NA | NA | NA | NA |
| 5% RH) | Example 2 | 0.33 | 0.16 | ND | 0.44 |
| | | | (RRT 0.21) | | |
| | Example 3 | 0.21 | ND | ND | 0.21 |
| | Example 4 | 0.27 | ND | ND | 0.27 |
| | Example 5 | | | | |
| 2M (25 ± 2° C./ | Example 1 | NA | NA | NA | NA |
| 60 ± 5% RH) | Example 2 | 0.67 | ND | ND | 0.67 |
| | Example 3 | | | | |
| | Example 4 | | | | |
| | Example 5 | | | | |
| 1M (2° C.-8° C.) | Example 1 | NA | NA | NA | NA |
| | Example 2 | ND | ND | ND | ND |
| | Example 3 | ND | ND | ND | ND |
| | Example 4 | ND | 0.07 | ND | 0.07 |
| | Example 5 | | | | |
| 2M (2° C.-8° C.) | Example 1 | NA | NA | NA | NA |
| | Example 2 | 0.12 | ND | ND | 0.12 |
| | Example 3 | | | | |
| | Example 4 | | | | |
| | Example 5 | | | | |

NA-Due to high impurity levels at Initial Stage, was not studied further
ND-Not detected
BDL-Below Detection Limit
*Awaited

Results: It is evident from Table 2 that total impurities (%) after storing for 1 month at 25±2 °C/60±5% RH are 0.21% in Example 3 (with polysorbate and cyclodextrin) and no impurities were detected at 2 °C-8 °C. The composition 3 (with polysorbate and cyclodextrin) and composition 5 (polysorbate with citrate buffer) when subjected to stress studies at 40±2 °C/NMT 25% RH for 4 weeks, total impurities were found to be 0.60% and 0.61% respectively and in composition 2 (with tyloxapol) was found to be 1.92%. This indicates that selection of specific buffer and stabilizing agent plays a crucial role in providing the stability to the composition of the present invention. This is evident from the above results that compositions comprising either polysorbate as stabilizing agent along with cyclodextrin or polysorbate as stabilizing agent along with phosphate or citrate buffer were found to be more stable.

We claim:

1. An ophthalmic suspension composition comprising:
brinzolamide or a salt thereof;
latanoprost or a salt thereof;
a stabilizer; and
one or more pharmaceutically acceptable excipients.

2. The composition as claimed in claim 1, wherein the composition is administered once daily.

3. The composition as claimed in claim 1, wherein the ratio of brinzolamide or a salt thereof:latanoprost or a salt thereof:stabilizer is 200:1:40-125.

4. The composition as claimed in claim 3, wherein the ratio of brinzolamide or a salt thereof:latanoprost or a salt thereof:stabilizer is 200:1:40-80.

5. The composition as claimed in claim 4, wherein the ratio of brinzolamide or a salt thereof:latanoprost or a salt thereof:stabilizer is 200:1:40-60.

6. The composition as claimed in claim 1, wherein the composition comprises brinzolamide or a salt thereof in an amount of about 0.01% w/v to about 10% w/v of the composition.

7. The composition as claimed in claim 1, wherein the composition comprises latanoprost or a salt thereof in an amount of about 0.01% w/v to about 5% w/v of the composition.

8. The composition as claimed in claim 1, wherein the amount of stabilizer is 0.001% w/v to about 10.0% w/v, preferably from 0.1% w/v to 1.0% w/v of the composition.

9. The composition as claimed in claim 1, wherein the stabilizer is selected from the group comprising of cyclodextrin or its derivative, tyloxapol, polysorbate, Polyoxyethylene hydrogenated castor oil, polyoxyl 15 hydroxystearate or macrogol 15 hydroxystearate, Poly (ethyleneoxide)/poly(propyleneoxide) triblock copolymers, polyvinyl caprolactam-polyvinyl acetate-polyethylene glycol graft co-polymer, polyoxyethylene fatty acid esters, ethoxylated alkyl phenol, polyoxyethylene alkylphenyl ethers, and polyoxyethylene alkyl ethers and/or combinations thereof.

10. The composition as claimed in claim 9, wherein the stabilizer is selected from the group comprising of cyclodextrin or its derivative, polysorbate and/or combinations thereof.

11. The composition as claimed in claim 1, wherein the pharmaceutically acceptable excipients are selected from the group comprising of buffering agent, preservative, chelating agent, pH-adjusting agent, tonicity-adjusting agent, penetration enhancer, thickener/viscosity modifier/gelling agent, demulcents/humectant, polyol, polymers, solvent/co-solvent/vehicle and/or combinations thereof.

12. The composition as claimed in claim 11, wherein the buffering agent is selected from the group comprising of bicarbonate buffer, acetate buffer, citrate buffer, sodium phosphate monohydrate, sodium phosphate dihydrate, borate buffer, diethanolamine, triethanolamine or tromethamine buffer, histidine buffer, and/or combinations thereof.

13. The composition as claimed in claim 1, wherein the composition when stored for 6 months at 25 °C±2 °C/60%±5% RH, exhibits not more than 1-3% of total impurities.

14. The composition as claimed in claim 1, wherein the composition when stored for 6 months at 25 °C±2 °C/60%±5% RH, exhibits not more than 1.5% latanoprost acid.

15. The composition as claimed in claim 1, wherein the composition when stored for 1 month at 25 °C±2 °C/60%±5% RH, exhibits not more than 1% of total impurities.

16. The composition as claimed in claim 1, wherein the composition when stored for 1 month at 25 °C±2 °C/60%±5% RH, exhibits not more than 1.0% latanoprost acid.

17. The composition as claimed in claim 1, wherein the composition is used for the treatment of elevated intraocular pressure (IOP) in patients with ocular hypertension or open-angle glaucoma.

18. A method of treating elevated intraocular pressure (IOP) in patients with ocular hypertension or open-angle glaucoma by administering an ophthalmic suspension composition comprising of brinzolamide or a salt thereof and latanoprost or a salt thereof, stabilizer and one or more pharmaceutically acceptable excipients.
